(19) United States
(12) Patent Application Publication
(10) Pub. No.: US 2004/0204435 A1
(43) Pub. Date: Oct. 14, 2004

(54) Alternating treatment with topoisomerase I and topoisomerase II inhibitors

(76) Inventors: Joachim Liehr, Houston, TX (US); Beppino Giovanella, Houston, TX (US)

Correspondence Address: KILYK & BOWERSOX, P.L.L.C., 53 A EAST LEE STREET, WARRENTON, VA, 20186, US

(21) Appl. No.: 10/410,627

(22) Filed: Apr. 9, 2003

(51) Int. Cl. A61K 31/4745

(52) U.S. Cl. 514/283

(57) ABSTRACT

A method to treat cancer is described which involves alternating treatments with a topoisomerase-I inhibitor and a topoisomerase-II inhibitor. Other aspects of the invention are further described.

### BACKGROUND OF THE INVENTION

The present invention relates to the treatment of cancer and the like. More particularly, the present invention relates to an alternating therapy which is effective in treating all forms of cancer.

While there are many cancer drugs commercially available or in the experimental stage, no one drug seems to effectively treat cancer on a regular basis. In addition, while certain cancer drugs are effective, there appears to be evidence that the tumor becomes resistant to the cancer drug and then the once potent anti-cancer drug looses most of its effectiveness. Prior to the present invention, there was no effective way to address this problem.

Camptothecin (CPT), the parent compound of this family of chemicals, is a natural product isolated from a Chinese tree, Camptotheca acuminata.

CPT and its derivatives have potent anticancer activity. For instance, CPT and its derivatives 9-nitrocamptothecin (9NC) or 9-aminocamptothecin completely eradicate human tumor xenografts in nude mice. Only the lactone form of these drugs (shown below) has full anticancer activity. CPT was much less effective in cancer patients than in mice.

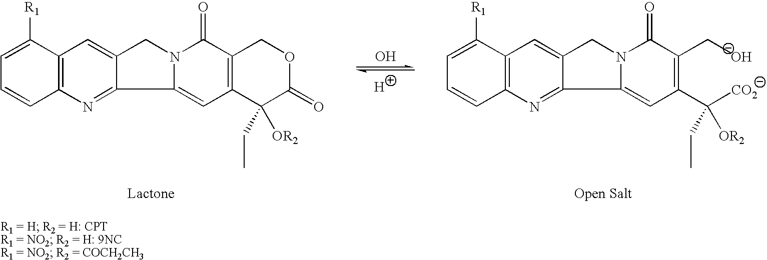

CPT lactone is insoluble in water, whereas the CPT carboxylate is soluble and thus is much easier to administer i.v. Unfortunately, CPT carboxylate has barely 10% of the anticancer activity of CPT, whereas toxicities are largely maintained. In the phase I and II trials conducted from 1970-1972 with the sodium salt of CPT, these toxicities have included myelosuppression, diarrhea and cystitis. Unfortunately, CPT showed no clinical benefit in these trials, because the largely inactive sodium salt was administered instead of the lactone.

9-Aminocamptothecin has undergone clinical trials, but it appears to be insufficiently effective to warrant further development. 9-Nitrocamptothecin has been tested against pancreas cancer and other cancers.

The mechanism of action of CPT is through topoisomerase I inhibition, and renewed interest in the drug has led to the development of a variety of analogs, some of which have higher potency than the parent drug. Some derivatives are water soluble such as Camptosar® (irinotecan, CPT-11) and Topotecan® which are currently approved for use in the USA for colon and ovarian cancers, respectively. Studies have shown that substitutions at the C-9 and C-10 positions enhance activity, and may confer water solubility. In general though, analogs that are water-soluble have reduced anti-cancer activity in preclinical models.

With CPT, diarrhea proved to be the dose-limiting toxicity. In phase I-II trials of 9NC its dose-limiting toxicity has been myelosuppression and gastrointestinal disturbances (nausea, vomiting, anorexia). Although 9NC is thought by some to be one of the best treatments for pancreatic cancer available today, it nevertheless is less effective against human pancreas tumors in humans than against human cancer xenografts in nude mice. A reason for the differential effects in humans and mice is the differential metabolism in these species. A species difference was observed also in the metabolism of 9NC in mice and humans that might explain the differential efficacies. Up to 50% of the administered dose circulates as active lactone form in mouse plasma, whereas in humans only 2-5% of the drug circulates as lactone and the remainder as inactive carboxylate salt. Human serum albumin (HSA) binds to the carboxylate salt of CPT or 9NC with an affinity exceeding 100-fold the binding of the lactone. As a result, the drug exists in human plasma almost exclusively in the inactive carboxylate form. In contrast, mouse serum albumin binds to the carboxylate forms of CPT or 9NC with much less affinity than HSA. Correspondingly, in mice the equilibrium between active lactone and salt forms of CPT or 9NC approaches 1:1. It was already known from previous studies that the lactones of CPT and its derivatives were pharmacologically much more active whereas the ring-opened salt forms carried only approximately 1/10 the chemotherapeutic activity of that of the lactone.

In response to this inactivation of drug by HSA, novel derivatives with stable lactone rings have also been developed. This stabilization of the lactone ring has been achieved by esterification of the 20-hydroxy group of 9NC by synthesizing propionate esters of CPT or 9NC and other esters as shown in the chemical structure above.

HSA binding inhibition has been accomplished for these ester derivatives by placing an ester substituent onto the C-20 hydroxy group of 9NC or CPT in the immediate vicinity of the lactone moiety to be protected. The ester function has been chosen to: a) inhibit binding to HSA and thus make the drug resistant to hydrolysis to the carboxylate form while in circulation; and b) permit ester hydrolysis to pharmacologically active 9NC lactone by tumor cell or tissue esterases.

In preclinical tests of 9-nitrocamptothecin 20(S)-propionate ester and of camptothecin 20(S)-propionate ester, results in cells in culture and in animals have shown these propionate esters to be excellent candidates for cancer treatment. Esterification of 9NC or CPT at the same time minimizes toxicity, because the ester itself may not be active, but is a prodrug. Thus, these compounds are at in part, activated by hydrolysis to 9NC or CPT lactone respectively, in tissues and less so while in circulation. This CPT or 9NC lactone is the pharmacologically active and also the toxic agent.

A second reason for the limited effectiveness of camptothecin-based therapy in humans compared to the potent anticancer activity in mice is based on the nature of topoisomerase expression. As noted above, CPT and its derivatives are inhibitors only of topoisomerase I. These compounds bind irreversibly to the enzyme, arrest DNA replication, and cause DNA strand breakage. In this way, they inhibit (tumor) cell replication.

It is important to note that CPT and its derivates are selective inhibitors only of topoisomerase I and do not affect tumors expressing topoisomerase II. Therefore, tumors expressing topoisomerase II will be resistant to treatment by camptothecins and will continue to grow. This resistance of topoisomerase II dependent tumors to camptothecin treatment may be evident from the beginning of chemotherapy as a certain % age of tumor cells may express topoisomerase I, and the remainder may express topoisomerase II. Alternatively, tumors may express only topoisomerase I in the initial stages of chemotherapy and respond to treatment, but then become resistant to camptothecin-based therapy, because tumor cells may have switched completely or partially to expressing topoisomerase II. Such biochemical changes in enzyme expression by tumors are known to exist.

Several inhibitors of topoisomerase II have been identified and are available on the market for tumor treatment. One of the least toxic and more effective compounds used for topoisomerase II-dependent cancer growth inhibition is VP-16 (etoposide). This compound has been available for some time and is usually used in combination with other cancer drugs. The main beneficial effect of VP-16 treatment may be the conversion of topoisomerase II-expressing tumors into expression of topoisomerase I. Thus, VP-16 acts not so much by killing growing cancer cells due to its inhibiting action on topoisomerase II. Rather it acts as a sensitizer and converts cells to express topoisomerase I.

Accordingly, there is significant room for improvement in the treatment of cancer.

### SUMMARY OF THE PRESENT INVENTION

A feature of the present invention is to provide a method to treat cancer.

Another feature of the present invention is to provide methods to sensitize a patient to Topo-I treatment.

A further feature of the present invention is to provide a method to combat a patient's resistance to chemotherapy.

A further feature of the present invention is to provide an improved method of treatment compared to the sole administering of Topo-I inhibitors.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates to a method to treat cancer in a patient, wherein the method includes administering a first formulation containing a Topo-I inhibitor and then administering a second formulation containing a Topo-II inhibitor.

The present invention further relates to a method which re-starts the administering of the first formulation after administering the second formulation and optionally repeating this alternating treatment one or more times.

The present invention further relates to a method to sensitize a patient to Topo-I inhibitor treatment by administering a formulation containing a Topo-II inhibitor and then afterwards administering a formulation containing a Topo-I inhibitor and optionally repeating this sequence of alternating treatment one or more times.

The present invention further relates to a kit which contains a formulation containing a Topo-I inhibitor and a formulation containing a Topo-II inhibitor.

It is to be understood that both the foregoing general description and the following detailed description are exemplary and explanatory only and are intended to provide a further explanation of the present invention, as claimed.

The accompanying drawings, which are incorporated in and constitute a part of this application, show aspects of the present invention, and together with the description, serve to explain the principals of the present invention.

### BRIEF DESCRIPTION OF THE DRAWINGS

FIG. 1 is a graph representing a representative treatment of a patient showing the timing or sequence of administering a Topo-I inhibitor and Topo-II inhibitor and the repeating of these alternating treatments.

### DETAILED DESCRIPTION OF THE PRESENT INVENTION

The present invention relates to a method to treat cancer in a patient. The method involves using a combination of Topo-I and Topo-II inhibitors in an alternating fashion. In more detail, the present invention relates to a method to treat cancer in a patient which involves administering a first formulation containing a Topo-I inhibitor and then administering a second formulation containing a Topo-II inhibitor. It is preferred to first administer the first formulation containing the Topo-I inhibitor. There is no necessity to administer this first formulation and one can easily start with the second formulation first and then administer the first formulation afterwards. Preferably, the administering of the first formulation and the second formulation is done sequentially or in an alternating fashion. In other words, there is preferably no overlap of administering each formulation.

Generally, the first formulation containing the Topo-I inhibitor is administered to a patient until resistance to the Topo-I inhibitor develops. For instance, this resistance can be seen if the tumor does not shrink or reduce in size any more. This can also be done using cancer markers such as CA19-9 or other conventional tumor markers. By measuring such tumor markers, one can readily see when the reduction in the tumor size has essentially ceases and the tumor is beginning to grow in size again. At this point, it is preferred to stop treatment with the formulation containing the Topo-I inhibitor and then begin treatment with the formulation containing the Topo-II inhibitor.

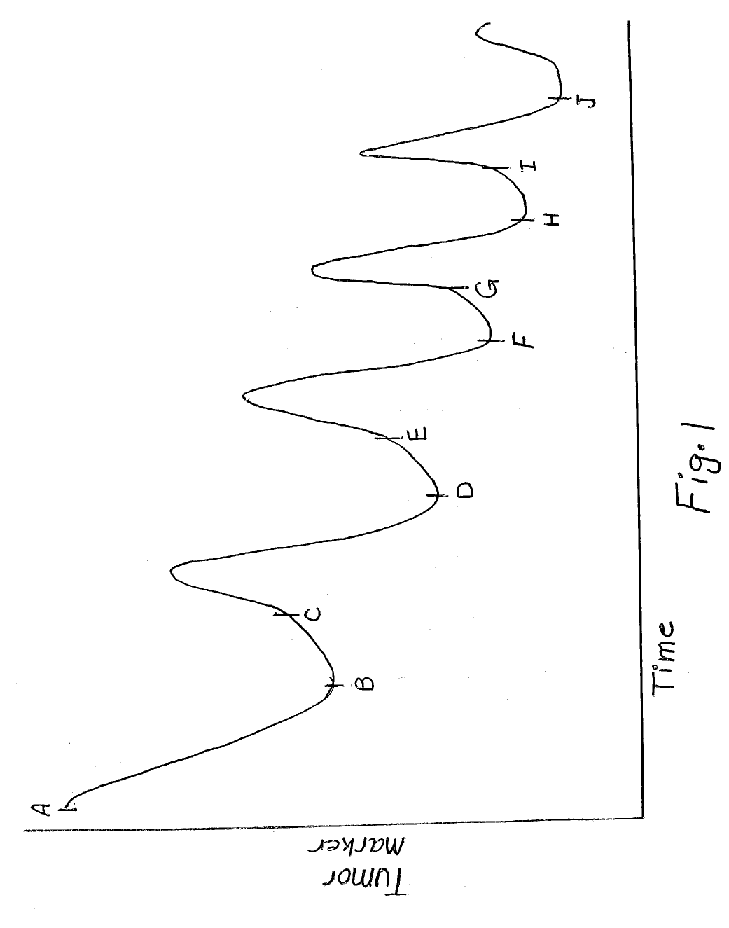

Generally, the formulation containing the Topo-II inhibitor is administered to the patient until the tumor begins to grow sharply. Another way to determine when to stop administering the formulation containing the Topo-II inhibitor is when the rise in the tumor growth (e.g., rate of growth) as referenced by the measuring discussed above substantially matches the decrease in tumor growth (e.g., rate of reduction) resulting from the most previous administering of the formulation containing the Topo-I inhibitor. This can be shown, just as an example, in FIG. 1 where the Topo-I administering is shown as well as the administering of the Topo-II inhibitor and the alternating treatments. Another way to determine when to alternate treatments is taking a biopsy of the tumor to measure Topo-I and/or Topo-II amounts. However, with most patients this is not readily feasible from the standpoint of taking many biopsies. Another manner to determine alternating treatment is to simply rely on the amount of time that the Topo-I inhibitor was administered and then to treat with the Topo-II inhibitor for substantially the same time or exactly the same time or within 25% of this time. For instance, if the formulation containing the Topo-I inhibitor was administered for five days, then the formulation containing the Topo-II inhibitor would be administered for five days, or about five days, or five days±25% of this time which in other words would be from about three and a half days to about six and a half days.

Another means to determine the timing for the alternating treatments is to take a mouse such as a nude mouse and to graft the actual tumor cell of the patient on the mouse using xenograph technology. This provides an excellent model to determine the behavior of the tumor in the patient and to readily see the timing necessary for the most effective treatment with respect to alternating between each formulation.

With respect to the method of the present invention, the alternating treatment can involve an embodiment where there are certain times where neither treatment is provided. For instance, the formulation containing the Topo-I inhibitor can be administered for five continuous days and then before the formulation containing the Topo-II inhibitor is administered the patient can have no treatment for one day, two days, three days, or more.

Also, in another embodiment, the formulation containing the Topo-I inhibitor can be administered for an exact time such as four days and then the formulation containing the Topo-II inhibitor can be administered for four days and so on maintaining a very exact sequence of administrations of each formulation. While this method can be effective, it is not the most effective means for treatment since it does not take into account the ever-changing state of the tumor. Ideally, the most effective treatment is to alternate treatments at the most optimal time as described above which provides the best means to stay on top of the tumor growth and to progressively reduce the tumor size over time without letting the tumor gain in resistance or growth.

In one embodiment of the present invention, the present invention involves the biochemical conversion of tumors expressing Topoisomerase II to cells expressing only Topoisomerase I as a result of treatment with one or more Topoisomerase II inhibitors.

This biochemical conversion sensitizes tumors to treatment by the much more potent topoisomerase I inhibitors, such as, camptothecin-based anticancer chemotherapy, like 9-nitrocamptothecin or esters such as 20-propionate esters of 9-nitrocamptothecin or camptothecin. Once tumors grow resistant to Topo-I inhibitors, like CPT-based therapy by switching back to expressing topoisomerase II, tumors are re-treated with Topo-II inhibitors, like VP-16 until such time that all tumor cells have been converted again to express topoisomerase I. At that point they will be treated again with one or more Topo-I inhibitors, like 9-nitrocamptothecin or 20-propionate esters of 9-nitrocamptothecin or camptothecin. The present invention permits a back and forth (ping pong type) alternating treatment with either a Topo-I inhibitor, like camptothecin-based therapy (e.g., 9-nitrocamptothecin, 20-propionate esters of 9-nitrocamptothecin or camptothecin) or a Topo-II inhibitor, like VP-16. The Topo-II inhibitors, like VP-16 sensitizes tumors to Topo-I inhibitors, like camptothecin-based therapy by biochemical conversion of tumor cells to express topoisomerase I instead of topoisomerase II.

The main killing of tumor cells and the shrinkage of the tumor is subsequently achieved by treating with potent camptothecin-based therapy or other Topo-I inhibitors. An optimal back-and-forth alternating treatment with a Topo-I inhibitor, like camptothecin-based therapy and then with a Topo-II inhibitor, like VP-16 will result in a step-wise reduction of tumor load.

The sensitization of topoisomerase II-dependent tumors by treating with VP-16 or other active agents, and the subsequent conversion to expression of topoisomerase I results in the elimination of resistance to Topo-I inhibitors, like camptothecin-based cancer chemotherapy. With most anticancer agents, resistance to drug treatment cannot be overcome once tumors have stopped responding to chemotherapy. The present invention re-sensitizes tumors to start responding again to topoisomerase I-inhibiting treatment, such as camptothecin-based therapy. The biochemical basis for this re-sensitizing is the fact that tumors express only either topoisomerase I or II. Other isoforms of this enzyme are not known. Therefore, the biochemical conversion of tumor cells to express topoisomerase I from previously expressing isoform II represents a re-sensitization to the potent tumor growth inhibition by camptothecin-base chemotherapy.

As shown, for instance, in FIG. 1, this graph represents one example of the timing of the administering of the Topo-I inhibitor and the administering of the Topo-I inhibitor. At point A in FIG. 1, this would be representative of the beginning of treatment in a patient having cancer. At this point, the tumor markers are quite elevated in the patient. Point A would be the approximate beginning of the administering of a Topo-I inhibitor. By administering a Topo-I inhibitor, the tumor size is reduced and the tumor marker count is decreased. Point B would be a suitable time to stop administering the Topo-I inhibitor since the patient is beginning to show no response to the Topo-I inhibitor. Point B is also a suitable time to begin administering the Topo-II inhibitor in order to re-sensitize the tumor to Topo-I inhibitors. Point C in FIG. 1 is an approximate time when to stop treatment of the Topo-I inhibitor because the tumor is beginning to grow rapidly once again. At point C, the administering of the Topo-I inhibitor, which can be the same or different from the previous treatment, can again be administered. By the time the Topo-I inhibitor is effective, the tumor will initially grow and once the Topo-I inhibitor is effective, the tumor growth as measured by the tumor marker will begin to peak out and rapidly decrease as shown in the slope between point C and point D in FIG. 1. At point D, decrease in the tumor growth is beginning to slow down thus showing that the tumor has once again shown a resistance to Topo-I inhibitor treatment. At point D, the stopping of the Topo-I inhibitor treatment would be suitable and the beginning of the Topo-II inhibitor treatment would be effective. At point E in FIG. 1, again a sharp rise in tumor growth is beginning to be seen and therefore a change from Topo-II inhibitor treatment to Topo-I treatment would be appropriate. This same sequence of alternating treatments can proceed as shown in FIG. 1 wherein points G and I would be appropriate times to stop Topo-II inhibitor treatment and begin Topo-I inhibitor treatment. Similarly, points F, H, and J would be appropriate times to stop Topo-I inhibitor treatment and being Topo-II inhibitor treatment. The goal with respect to this invention and as shown in FIG. 1 is to steadily decrease the size of the tumor over time which is what is shown in FIG. 1.

With respect to the Topo-I inhibitors, any drug (e.g., composition or compound) that is a Topo-I inhibitor can be used. Preferably, the Topo-I inhibitor is a very potent Topo-I inhibitor such as camptothecin-based chemotherapy. Weak Topo-I anti-cancer agents are not preferred such as topotecan. The camptothecin can be water-soluble or water-insoluble. Water-insoluble CPTs are preferred. The CPT can be camptothecin, a derivative thereof (e.g., ester thereof), and/or a prodrug of CPT.

Specific examples include those camptothecin-based compounds set forth in U.S. Pat. Nos. 6,407,239; 6,407,118; 6,352,996; 6,342,506; 6,228,855; 6,218,399; 6,166,029; 6,120,793; 6,096,336; 6,080,751; 5,968,943; 5,922,877; 5,889,017; 5,731,316; 5,652,244; 5,552,154. Other examples of Topo-I inhibitors include those set forth in U.S. Pat. Nos. 6,512,118; 6,497,896; 6,486,320; 6,465,008; 6,310,210; 6,291,676; 6,288,072; 6,242,457; 6,100,273; and U.S. patent application Ser. No. 10/139,817, filed May 6, 2002; Ser. No. 10/139,778, filed May 6, 2002.

Specific examples of suitable Topo-I inhibitors for purposes of the present invention include 20(S)-camptothecin; 9-nitro-20(S)camptothecin; Camptothecin 20-O-propionate; Camptothecin 20-O-butyrate; Camptothecin 20-O-valerate; Camptothecin 20-O-heptanoate; Camptothecin 20-O-nonanoate; Camptothecin 20-O crotonate; Camptothecin 20-O-2′,3′-epoxy-butyrate; 9-Nitrocamptothecin 20-O-acetate; 9-Nitrocatnptothecin-20-O-propionate; 9-Nitrocamptothecin 20-O-butyrate. Each of the U.S. patents and U.S. patent applications set forth above and any patent and/or publication mentioned throughout this application are incorporated in therein entirety by reference herein and form a part of the present application.

More specific examples of camptothecin derivatives are preferably water-insoluble aromatic camptothecin esters. The aromatic camptothecin esters preferably have the formula (I):

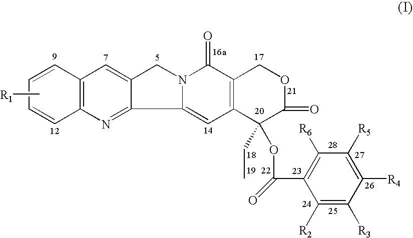

In this formula, the various R groups represent substituents on one of the rings of the structure above. In particular, R¹ represent H, NO₂, NH₂, N₃, a halogen (e.g., F, Cl, Br, I), carboxyl (COOH), a C₁₋₁₆ alkyl group, C₁₋₁₆ alkylenyl group, a C₃₋₈ cycloalkyl group, a C₁₋₈ alkoxyl group, an aroxyl group, CN, SO₃H, a C₁₋₈ halogenated alkyl group, (CH₂)ₙNR₂⁷ (where R⁷ is H, or a C₁₋₈ alkyl group, n is an integer of from 1 to about 8), hydroxyl, SH, SR⁸ (where R⁸ is a C₁₋₈ alkyl group, or a phenyl group, or a substituted phenyl group), a carbonyl group, (e.g., COR⁹, where R⁹ is a C₁₋₈ alkyl group, or a phenyl group, or a substituted phenyl group), a SiR₃¹⁰ (where R¹⁰ is a C₁₋₄ alkyl group). The R¹ group is respectively positioned at the 9, or 10, or 11, or 12 position of ring A. R¹ can also be a disubstituted 10,11-O—(CH₂)_{y}—O— group (where y is an integer of from 1 to 3). X represents H, a C₁₋₈ alkyl group, a C₁₋₈ alkenyl group, a C₁₋₈ alkoxyl group, an aroxyl group, a SiR₃¹¹ group (where R¹¹ is a C₁₋₄ alkyl group), or CH₂NZY where Z and Y are, independently, H, C₁₋₄ alkyl, or a C₁₋₄ halogenated alkyl group. R², R³, R⁴, R⁵, and R⁶ are, independently, H(s), C₁₋₁₂ alkyl group(s), C₁₋₁₂ alkenyl group(s), COOH(s), SO₃H(s), CN(s), CF₃(s), CCl₃(s), CH₂F(s), CH₂Cl(s), CHF₂(s), CHCl₂(s), OH(s), OR¹²(s) (where R¹² is a C₁₋₈ alkyl group, or a C₁₋₈ alkenyl group, or an aromatic group), N₃, NO₂(s), NR₂¹³(s) (where R¹³ is H, or C₁₋₄ alkyl group), carbonyl group (s), halogen(s).

More preferred aromatic camptothecin esters of the present invention are as follows, wherein:

| | |
|---|---|
| R² = R³ = R⁴ = R⁵ = R⁶ = H; | R² = R³ = R⁵ = R⁶ = H, R⁴ = CF₃; |
| R² = R³ = R⁴ = R⁶ = H, R⁵ = CF₃; | R² = R³ = R⁴ = R⁵ = H, R⁶ = CF₃; |
| R² = R³ = R⁶ = H, R⁴ = R⁵ = NO₂; | R² = R⁴ = R⁶ = H, R³ = R⁵ = NO₂; |
| R² = R³ = R⁵ = H, R⁴ = R⁶ = NO₂; | R² = R³ = R⁵ = R⁶ = H, R⁴ = NO₂; |
| R² = R³ = R⁴ = R⁶ = H, R⁵ = NO₂; | R³ = R⁴ = R⁵ = R⁶ = H, R² = NO₂; |
| R³ = R⁴ = R⁵ = R⁶ = H, R² = CN; | R² = R³ = R⁴ = R⁶ = H, R⁵ = CN; |
| R² = R³ = R⁵ = R⁶ = H, R⁴ = CN; | R³ = R⁴ = R⁵ = R⁶ = H, R² = F; |
| R² = R³ = R⁴ = R⁶ = H, R⁵ = F; | R² = R³ = R⁵ = R⁶ = H, R⁴ = F; |
| R³ = R⁴ = R⁵ = R⁶ = H, R² = Cl; | R² = R³ = R⁴ = R⁶ = H, R⁵ = Cl; |
| R² = R³ = R⁵ = R⁶ = H, R⁴ = Cl; | R³ = R⁴ = R⁵ = R⁶ = H, R² = Br; |
| R² = R³ = R⁴ = R⁶ = H, R⁵ = Br; | R² = R³ = R⁵ = R⁶ = H, R⁴ = Br; |
| R³ = R⁴ = R⁵ = R⁶ = H, R² = OH; | R² = R³ = R⁴ = R⁶ = H, R⁵ = OH; |
| R² = R³ = R⁵ = R⁶ = H, R⁴ = OH. | |

Preferably for these above-mentioned preferred compounds, R¹ is hydrogen and X represents hydrogen as well.

Another group of preferred compounds are the following, wherein:

| | |
|---|---|
| R² = R³ = R⁴ = R⁵ = R⁶ = H; | R² = R³ = R⁵ = R⁶ = H, R⁴ = CF₃; |
| R² = R³ = R⁴ = R⁶ = H, R⁵ = CF₃; | R² = R³ = R⁴ = R⁵ = H, R⁶ = CF₃; |
| R² = R³ = R⁶ = H, R⁴ = R⁵ = NO₂; | R² = R⁴ = R⁶ = H, R³ = R⁵ = NO₂; |
| R² = R³ = R⁵ = H, R⁴ = R⁶ = NO₂; | R² = R³ = R⁵ = R⁶ = H, R⁴ = NO₂; |
| R² = R³ = R⁴ = R⁶ = H, R⁵ = NO₂; | R³ = R⁴ = R⁵ = R⁶ = H, R² = NO₂; |
| R³ = R⁴ = R⁵ = R⁶ = H, R² = CN; | R² = R³ = R⁴ = R⁶ = H, R⁵ = CN; |
| R² = R³ = R⁵ = R⁶ = H, R⁴ = CN; | R³ = R⁴ = R⁵ = R⁶ = H, R² = F; |
| R² = R³ = R⁴ = R⁶ = H, R⁵ = F; | R² = R³ = R⁵ = R⁶ = H, R⁴ = F; |
| R³ = R⁴ = R⁵ = R⁶ = H, R² = Cl; | R² = R³ = R⁴ = R⁶ = H, R⁵ = Cl; |
| R² = R³ = R⁵ = R⁶ = H, R⁴ = Cl; | R³ = R⁴ = R⁵ = R⁶ = H, R² = Br; |
| R² = R³ = R⁴ = R⁶ = H, R⁵ = Br; | R² = R³ = R⁵ = R⁶ = H, R⁴ = Br; |
| R² = R⁶ = H, R³ = R⁵ = NO₂, | R³ = R⁴ = R⁵ = R⁶ = H, R² = OH; |
| R⁴ = CH₃; | |
| R² = R³ = R⁴ = R⁶ = H, R⁵ = OH; | R² = R³ = R⁵ = R⁶ = H, R⁴ = OH. |

For the above second group of preferred compounds, R¹ is preferably 9-NO₂ and X represents hydrogen.

Another group of preferred compounds are the following, wherein:

| | |
|---|---|
| R² = R³ = R⁴ = R⁵ = R⁶ = H; | R² = R³ = R⁵ = R⁶ = H, R⁴ = CF₃; |
| R² = R³ = R⁴ = R⁶ = H, R⁵ = CF₃; | R² = R³ = R⁴ = R⁵ = H, R⁶ = CF₃; |
| R² = R³ = R⁶ = H, R⁴ = R⁵ = NO₂; | R² = R⁴ = R⁶ = H, R³ = R⁵ = NO₂; |
| R² = R³ = R⁵ = H, R⁴ = R⁶ = NO₂; | R² = R³ = R⁵ = R⁶ = H, R⁴ = NO₂; |
| R² = R³ = R⁴ = R⁶ = H, R⁵ = NO₂; | R³ = R⁴ = R⁵ = R⁶ = H, R² = NO₂; |
| R³ = R⁴ = R⁵ = R⁶ = H, R² = CN; | R² = R³ = R⁴ = R⁶ = H, R⁵ = CN; |
| R² = R³ = R⁵ = R⁶ = H, R⁴ = CN; | R³ = R⁴ = R⁵ = R⁶ = H, R² = F; |
| R² = R³ = R⁴ = R⁶ = H, R⁵ = F; | R² = R³ = R⁵ = R⁶ = H, R⁴ = F; |
| R³ = R⁴ = R⁵ = R⁶ = H, R² = Cl; | R² = R³ = R⁴ = R⁶ = H, R⁵ = Cl; |
| R² = R³ = R⁵ = R⁶ = H, R⁴ = Cl; | R³ = R⁴ = R⁵ = R⁶ = H, R² = Br; |
| R² = R³ = R⁴ = R⁶ = H, R⁵ = Br; | R² = R³ = R⁵ = R⁶ = H, R⁴ = Br; |
| R³ = R⁴ = R⁵ = R⁶ = H, R² = OH; | R² = R³ = R⁴ = R⁶ = H, R⁵ = OH; |
| R² = R³ = R⁵ = R⁶ = H, R⁴ = OH. | |

For this third group of preferred compounds, R¹ is preferably 9-NH₂ and X represents hydrogen.

For the above-described substitutents, preferred alkyl groups are —CH₃, —CH₂CH₃, CH₃CH₂CH₂—, CH₃(CH₂)₃—, CH₃(CH₂)₄—, CH₃(CH₂)₅—, and CH₃(CH₂)₆₋₁₇—, (CH₃)₂CH—, CH₃

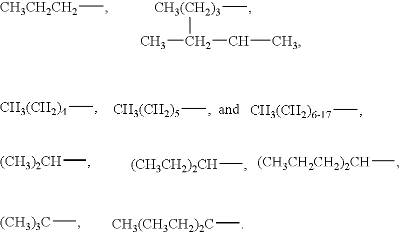

—CH—CH₃, (CH₃CH₂)₂CH—, (CH₃CH₂CH₂)₂CH—, (CH₃)₃C—, CH₃(CH₃CH₂)₂C—.

Preferred alkylenyl groups are CH₂═CH—, CH₃CH═CH—, CH₃CH═C(CH₃)—, CH₃CH═CHCH₂—, CH₃CH₂CH═CHCH₂—, CH₃(CH₂)₃₋₁₅CH═CH—, CH₃CH═CH—(CH₂)₃₋₁₅CH₂—, CH₂═CH—CH═CH—, CH₃CH═CH—CH═CH—, CH₃(CH₂)₃₋₆—CH═CH—CH═CH—(CH₂)₃₋₆—CH₂—.

Preferred C₃₋₈ cycloalkyl groups are

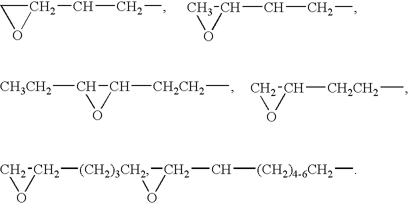

Preferred C₁₋₈ alkoxyl groups are

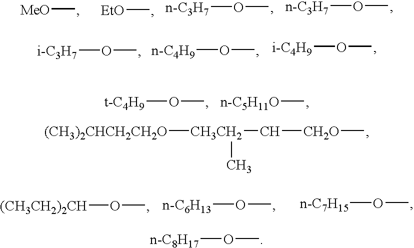

Preferred aroxyl groups are p-CH₃OC₆H₄—, m-CH₃O—C₆H₄—, o-CH₃OC₆H₄—, o,p-Dimethoxyl phenyl-, m,m-Dimethoxyl phenyl-, m,p-Dimethoxyl phenyl-, o-CH₃CH₂OC₆H₄—, m-CH₃CH₂OC₆H₄—, p-CH₃CH₂O—C₆H₄—.

Preferred cycloalkyl groups are cyclo-C₃, cyclo-C₄, cyclo-C₅, cyclo-C₆, cyclo-C₇, cyclo-C₈, alkyl substituted cyclo-C₃, alkyl substituted cyclo-C₄, alkyl substituted cyclo-C₅, alkyl substituted cyclo-C₆, alkyl substituted cyclo-C₇, and alkyl substituted cyclo-C₈ (where alkyl includes preferably those alkyl groups described above).

Preferred unsubstituted and substituted phenyl groups are C₆H₅—, (o,m,p) CH₃C₆H₄—, halogen substituted phenyl groups (X C₆H₄, wherein X═F, Cl, Br, I), (o,p,m) CH₃OC₆H₄—, (o,m,p) NO₂C₆H₄—, (o,m,p) NH₂C₆H₄—, (o,m,p) CNC₆H₄—.

Preferred carbonyl groups are

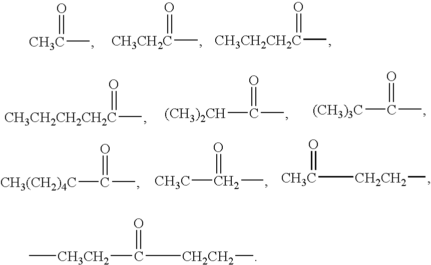

Other CPT derivatives are characterized by formula II shown below:

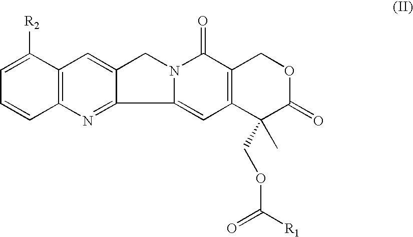

wherein R₂ is H, NH₂, or NO₂. R₁ in formula II represents a C₁-C₁₅ alkyl group, a C₃-C₈ cycloalkyl group, a C₂═C₁₅ alkenyl group or a C₂-C₁₅ epoxy group. Preferably, when R₂ is H, R₁ is CH₂CH₃; CH₂CH₂CH₃; CH₂CH₂CH₂CH₃; CH₂CH₂CH₂CH₂CH₂CH₃; CH₂CH₂CH₂CH₂CH₂CH₂CH₂CH₃; CH═CH₂; CH═CHCH₃ (trans); or

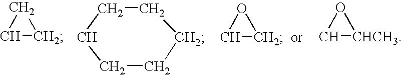

Also, when R₂ is NO₂, R₁ is preferably CH₃; CH₂CH₃; or CH₂CH₂CH₃; CH═CH₃; CH═CHCH₃(trans); CH₃; CH₂CH₃; or CH₂CH₂CH₃; CH═CH₃; CH═CHCH₃(trans); or

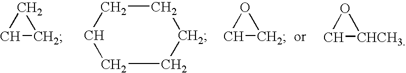

With respect to the Topo-II inhibitors, any conventional Topo-II inhibitors such as VP-16 can be used. Other examples of Topo-II inhibitors include, but are not limited to, Amsacrine(IT-AMSA); and VM-26-Teniposde See Belani C P, Doyle L A, Aisher J: Etoposide: Current status and future perspectives in the management of malignant neoplasms. Cancer Chemother. Pharmacol. 34: 5118, 1994; Blume K G, Long G D, Negrin R S, et al.: Role of etoposide (VP16) in preparatory regimens for patients with leukemia or lymphoma undergoing allogeneic bone marrow transplantation. Bone Marrow Transplant. 14:59, 1994; Inde D C: Chemotherapy of lung cancer. N. Engl. J. Med. 327:1434, 1992; Richie J P: Detection and treatment of testicular cancer. CA Cancer J. Clin. 43:151, 1993; Bjorkholm M: Etoposide and teniposide in the treatment of acute leukemia. Med. Oncol. Tumor Phar. 3:7, 1990; Oishi N, Berenberg J, Blumenstein B A, et al.: Teniposide in metatastic renal and bladder cancer-A Southwest oncology group study. Cancer Treat. Rep. 71:1307, 1987; Lokich J J: Teniposide (VM26) as a single drug treatment for patients with extensive small cell lung carcinoma-A phase II study of the southwest oncology group. Cancer 82:993, 1998; Brandes A A, Rigon A, Zampieri P, et al.: Carboplatin and teniposide concurrent with radiotherapy in patients with glioblastoma multiforme-a phase I study. Cancer 82:355, 1998; Arlin Z A, Ahmed T, Mittelman A, et al.: A new regimen of amsacrine with high dose cytarabine is safe and effective therapy for acute leukemia. J. Clin. Oncol. 5:371, 1987; Jehn V, Giller S, Hinrichs F, et al.: Five day 4′(9acridinylamino) methanesulphon-M-anisidine and intermediate dose cytosine arabinoside in high risk relapsing or refractory acute myeloid leukemia. J. Cancer Res. Clin. Oncol. 117:489, 1991; Hayath M, Ostronoff M, Gilles E, et al. Salvage therapy with methyl-gag, high dose ARA-C, M-AMSA and Ifosfamide (MAMI) for recurrent or refractory lymphoma. Cancer Invest. 8:1, 1990; Cassileth P A, Gale R P: Amsacrine: A Review. Leuk. Res. 10:1257, 1986. All of these references are incorporated in their entirety by reference herein.

The treatment of the present invention is effective in the treatment of cancer, including, but not limited to, malignant tumors and other forms of cancer. As used herein, the term malignant tumor is intended to encompass all forms of human carcinomas, sarcomas, and melanomas which occurs in the poorly differentiated, moderately differentiated, and well differentiated forms. In administering the compounds of the present invention to patients in need of such treatment, an effective amount of the compound or formulation containing one or more compounds of the present invention is administered to the patient. As used herein, an “effective amount” of the compound of the present invention is intended to mean that amount of the compound which will inhibit the growth of, or retard cancer, or kill cancer or malignant cells, and/or cause the regression and/or palliation of cancer such as malignant tumors, i.e., reduce the volume or size of such tumors or eliminate the tumor entirely. On an effective amount in the case of the Topo-II inhibitor, is an amount capable of restoring effectiveness of the Topo-I inhibitor.

The compounds used in the present invention and formulations used in the present invention can be used in the treatment of a number of tumors and/or cancers including, but not limited to, human cancers of the lung, breast, colon, prostate, melanoma, pancreas, stomach, liver, brain, kidney, uterus, cervix, ovaries, urinary track, gastrointestinal, and other solid tumors which grow in a anatomical site other than the blood stream as well as blood borne tumors such as leukemia. Other solid tumors include, but are not limited to, colon and rectal cancer.

The compounds or formulations used in the treatment of the present invention can be administered by any acceptable route including, but not limited to, orally, intramuscularly, transdermally, intravenously, through an inhaler or other air borne delivery systems, and the like. Preferably, the compounds and the formulations used in the present invention are administered orally, intramuscularly, or transdermally and most preferably delivered orally. Examples of transdermally delivery systems can be found, for instance in U.S. Pat. Nos. 5,552,154 and 5,652,244 incorporated in their entirety by reference herein. The compounds or formulations of the present invention can also be administered to a patient through a liposome system such as ones described in U.S. Pat. Nos. 5,882,679; 5,834,012; 5,783,211; 5,718,914; 5,631,237; 5,552,156; 5,059,421; 5,000,958; 5,874,105; 5,567,434; 5,549,910; 5,043,165; 5,736,156; 5,567,433; and 4,663,161, all incorporated in their entirety by reference herein.

In addition, the compounds and formulations used in the present invention can be used in combination with other drugs and formulations for the treatment of cancers such as taxol, taxotere, or their derivatives as well as cisplatin and derivatives thereof

With mammals, including humans, the effective amounts for each inhibitor formulation can be administered on the basis of body surface area. The interrelationship of dosages for animals of various sizes, species and humans (based on mg/M² of body surface) is described by E. J. Freireich et al., Cancer Chemother. Rep., 50(4):219 (1966). Body surface area may be approximately determined from the height and weight of an individual (see, e.g., Scientific Tables, Geigy Pharmaceuticals, Ardsley, N.Y., pp. 537-538 (1970). An effective amount of the camptothecin compounds in the present invention can range from about 12.5 mg/m² of body surface per day to about 31.3 mg/m² of body surface per day.

The preferred effective amounts or dosages of the Topo-I inhibitors, such as CPT, and derivatives thereof in mice are about 1 to about 4 mg per/kg of body weight twice a week for an intramuscular route and about 0.75 to about 1.5 mg per/kg/day for the oral route. Effective amounts or dosages in mice are, for instance about 1.5 mg/Kg/week to about 10 mg/Kg/week for the transdermal route. For all of the administering routes, the exact timing of administration of the dosages can be varied to achieve optimal results. Generally, when using Intralipid 20 as the carrier for the compound, the actual dosage of the compound reaching the patient may be less. This is due to some loss of the compound on the walls of the syringes, needles, and preparation vessels, which is prevalent with the Intralipid 20 suspension. When a carrier, such as cottonseed oil is used, the above-described loss is not so prevalent because the compound does not adhere as much to the surfaces of syringes, and the like. For instance, and preferably, it has been found that generally about 2.5 mg compound per kg of body weight twice per week using cottonseed oil, administered by an intramuscular route, will deliver the same amount to the patient as 4.0 mg per/kg of body weight twice per week using Intralipid 20 as a carrier. Generally, about 1 mg to about 4 mg of the compound is added to about 0.1 ml to about 1 ml of carrier. Levels of the compounds were well tolerated by mice in the examples set forth below without weight loss or other signs of toxicity. These dosages have been administered for up to six months continuously without any ill effect.

Another important feature of the method provided by the present invention relates to the relatively low or no apparent overall toxicity of the Topo-I inhibitors, especially the specific camptothecin compounds mentioned and administered in accordance herein. Overall toxicity can be judged using various criteria. For example, loss of body weight in a subject over 10% of the initially recorded body weight (i.e., before treatment) can be considered as one sign of toxicity. In addition, loss of overall mobility and activity and signs of diarrhea or cystitis in a subject can also be interpreted as evidence of toxicity.

The compounds and formulations used in the present invention may be administered in combination with pharmaceutically acceptable carriers or dilutents, such as Intralipid 10 or 20 or natural oils, or other suitable emulsifiers for lipophilic compounds.

With respect to the use of Topo-II inhibitors, in order to achieve the desirable affect of biochemical conversion of topoisomerase II-expressing tumors to cells expressing isoform I preferably involves continuous exposure to intermediate doses of the Topo-II inhibitor. Such treatment can be achieved using, for instance, VP-16 taken orally and given every week continuously for five days at a dose of from about 50 to about 100 mg VP-16 followed by two treatment-free days. Generally, this treatment is repeated for five to six weeks to achieve full conversion of topoisomerase II to the I isoform. Of course, other dosages and time periods can be used as described above with respect to determining when the tumor begins to grow sharply or when the tumor growth rises substantially in the same growth patter as the tumor reduction scene with the Topo-I inhibitor.

Generally, as an example, the Topo-I inhibitor is administered for 1-4 weeks to the patient and then the Topo-II inhibitor is administered for about the same time, as described earlier.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof

What is claimed is:

1. A method to treat cancer in a patient comprising administering a first formulation comprising a topoisomerase-I inhibitor and then administering a second formulation comprising a topoisomerase-II inhibitor.

2. A method to treat cancer in a patient comprising alternating treatment between a first formulation comprising a topoisomerase-I inhibitor and a second formulation comprising a topoisomerase-II inhibitor.

3. The method of claim 1, further comprising administering a first formulation after administering said second formulation and optionally repeating the sequence one or more times.

4. The method of claim 1, further comprising repeating administering said first formulation and said second formulation one or more times, wherein said first formulation or said second formulation, or both are the same or different from the first formulation and second formulation administered initially.

5. The method of claim 1, wherein said topoisomerase-I inhibitor comprises a camptothecin or derivative thereof or prodrug thereof.

6. The method of claim 1, wherein said topoisomerase-I inhibitor comprises 20(S)camptothecin, 9-nitro-20(S)camptothecin, or combinations thereof.

7. The method of claim 1, wherein said topoisomerase-I inhibitor comprises an ester of camptothecin.

8. The method of claim 1, wherein said topoisomerase-I inhibitor comprises an aromatic ester of camptothecin.

9. The method of claim 1, wherein said topoisomerase-I inhibitor comprises: 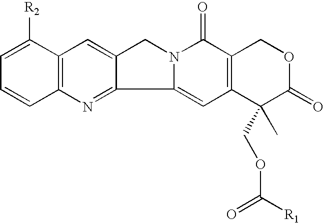
wherein R₂ is H, NH₂, or NO₂ and R₁ is a C₁-C₁₅ alkyl group, a C₃-C₈ cycloalkyl group, a C₂═C₁₅ alkenyl group or a C₂-C₁₅ epoxy group.

10. The method of claim 1, wherein said topoisomerase-I inhibitor comprises: 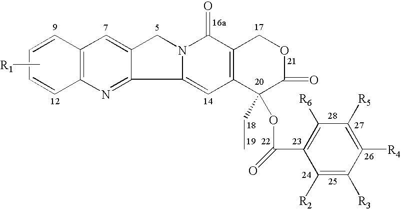
wherein R¹ represent H, NO₂, NH₂, N₃, a halogen, carboxyl (COOH), a C₁₋₁₆ alkyl group, C₁₋₁₆ alkylenyl group, a C₃₋₈ cycloalkyl group, a C₁₋₈ alkoxyl group, an aroxyl group, CN, SO₃H, a C₁₋₈ halogenated alkyl group, (CH₂)ₙNR₂⁷, hydroxyl, SH, SR⁸, a carbonyl group, a SiR₃¹⁰, and R², R³, R⁴, R⁵, and R⁶ are, independently, H(s), C₁₋₁₂ alkyl group(s), C₁₋₁₂ alkenyl group(s), COOH(s), SO₃H(s), CN(s), CF₃(s), CCl₃(s), CH₂F(s), CH₂Cl(s), CHF₂(s), CHCl₂(s), OH(s), OR¹²(s), N₃, NO₂(s), NR₂¹³(s) where R⁷ is H, or a C₁₋₈ alkyl group, and n is an integer of from 1 to about 8, where R⁸ is a C₁₋₈ alkyl group, or a phenyl group, or a substituted phenyl group, where R¹⁰ is a C₁₋₄ alkyl group, where R¹² is a C₁₋₈ alkyl group, or a C₁₋₈ alkenyl group, or an aromatic group, and where R¹³ is H, or C₁₋₄ alkyl group, carbonyl group(s), halogen(s).

11. The method of claim 1, wherein said cancer is human cancer of the lung, breast, colon, prostate, melanoma, pancreas, stomach, liver, brain, kidney, uterus, cervix, ovaries, urinary tract, gastral intestinal, other tumors which grown in an anatomical site other than the bloodstream, blood born tumors, colon, rectal, or combinations thereof.

12. The method of claim 1, wherein said first formulation, second formulation, or both is administered orally, intramuscularly, transdermally, intravenously, an air borne delivery system, or combinations therefore.

13. The method of claim 1, wherein said first formulation and said second formulation are administered orally.

14. The method of claim 1, wherein said first formulation is administered until resistance occurs to said formulation.

15. The method of claim 1, wherein said second formulation is administered after said resistance develops to said first formulation and said second formulation is administered until the growth rate of the cancer is substantially the same as the decrease rate in the cancer from the most recent treatment of said first formulation.

16. The method of claim 1, wherein said topoisomerase-II inhibitor comprises etoposide.

17. The method of claim 1, wherein said topoisomerase-I inhibitor is administered for from about 1 to about 4 weeks.

18. The method of claim 1, wherein said topisomerase-II inhibitor is administered for from about 1 to about 4 weeks.
